(19) United States
(12) Patent Application Publication
(10) Pub. No.: US 2013/0012760 A1
(43) Pub. Date: Jan. 10, 2013

(54) Magnetic Lumbar Orthosis with Rigid Support

(76) Inventor: Lev Tripolsky, Redwood City, CA (US)

(21) Appl. No.: 13/542,595

(22) Filed: Jul. 5, 2012

Related U.S. Application Data
(60) Provisional application No. 61/505,110, filed on Jul. 6, 2011.

(51) Int. Cl. A61N 2/00; A61F 5/00

(52) U.S. Cl. 600/15; 602/19

(57) ABSTRACT

A lumbar orthosis, comprising a rigid thermoplastic plate molded to the contours of the patient's back and at least one permanent magnet located on the orthosis that can provide magnetic therapy for patients with severe back problems, and further comprising adjustable supplementary fasteners that enable the patient to tighten or loosen the orthosis without removing it.

### CROSS-REFERENCE TO RELATED APPLICATIONS

The present application claims the benefit of U.S. provisional patent application No. 61/505,110, filed Jul. 6, 2011, which is incorporated herein by reference in its entirety.

### BACKGROUND

1. Field of Invention

The invention pertains to lumbar supports, specifically lumbar supports that use magnetic elements for therapeutic purposes.

2. Prior Art

Magnetic therapy is well known for its benefits in the treatment of musculoskeletal disorders. The application of magnets to a painful area is known to decrease pain and improve circulation. Since lower back pain is a significant problem for many people, there are several designs in the prior art for lumbar supports that incorporate magnets.

U.S. Pat. No. 4,480,596 to Shumiyashu discloses an elastic belt that fits around the lower back with magnets incorporated into it. The magnets are removably mounted to a flexible ABS sheet. The belt is relatively narrow, and thus, while it is useful in mild cases of back pain, it does not provide enough support for the back in cases of severe pain or instability.

U.S. Pat. No. 5,450,858 to Zablotsky et al. discloses an inflatable lumbar support that includes permanent magnets with a specified magnetic field pattern in order to improve circulation. The inflatable support provides a static stretch to the lower back muscles and improves back stability by providing compression. However, similarly to the Shumiyashu invention, the Zablotsky back support does not provide a sufficiently rigid orthosis for severe back pain or instability, which means that it can only provide magnetic therapy to patients with mild back pain.

### SUMMARY OF THE INVENTION

It is the primary object of this invention to provide a rigid lumbar orthosis that incorporates permanent magnets for magnetic therapy. This enables magnetic therapy to be used in cases of severe back pain or instability, which enables patients with more serious back problems to benefit from its effects.

In the preferred embodiment, the orthosis comprises a rigid backplate—a wide sheet of thermoplastic material that is molded to the patient's back and inserted into a pocket on the orthosis—and a semi-rigid front plate—a sheet of semi-rigid material that is located on the patient's abdomen and inserted into a pocket on the orthosis. The semi-rigid front plate is preferably more flexible than the rigid backplate, for greater comfort.

The permanent magnets may be laid out in a grid pattern, laid out in vertical arrays, or arranged in any other way on the area of the lumbar support that covers the patient's back. The magnets may be located on top of, within, or underneath the rigid backplate, or may be located on the surrounding areas of the lumbar orthosis so as not to interfere with the rigid backplate. In another embodiment of the invention, permanent magnets may also be located on the patient's abdominal area as well as on the patient's back. This creates a three-dimensional magnetic field that provides a greater therapeutic benefit to the patient.

Any type of magnet can be used for the present invention as long as the magnetic field it provides is sufficient for magnetic therapy use. In the preferred embodiment, disc-shaped magnets arranged in a rectangular array are used.

In another embodiment of the invention, the rigid backplate itself is made of a magnetic material such that it can provide a sufficient magnetic field for a therapeutic benefit while not requiring any additional magnets to be used. The front plate may also be magnetic.

Another embodiment of the invention further comprises supplementary fasteners in addition to the main fastener in the front of the orthosis, which enable the patient to make fine adjustments to the tightness of the orthosis without removing the orthosis. Since this orthosis is intended for patients with serious back problems, taking it off altogether may be dangerous for the patient. The adjustable side fasteners make it possible to keep the orthosis on throughout the day. The supplementary fasteners are preferably straps that are nonremovably attached to the belt on one end and that have a Velcro fastener on the other end that can be removably attached to the belt at any point on the belt.

In another embodiment of the invention, the belt is made of a heat-retention fabric that can provide therapeutic heat to the patient's back, complementing the magnetic therapy and further compounding the therapeutic value of the invention. The pocket for the rigid backplate or the pocket for the semi-rigid front plate may also be used for the insertion of a heat or cold pack, to provide heat/cold therapy for the patient.

The description herein provides preferred embodiments of the present invention, but should not be construed as limiting its scope. Variations in the type of rigid lumbar orthosis utilized, the type, shape, and quantity of the magnets used, the strength of the magnetic field, and the type of fasteners used for the orthosis, can all be incorporated into the present invention. The scope of the present invention should be determined by the appended claims and their legal equivalents, rather than the examples given.

### DESCRIPTION OF THE DRAWINGS

FIG. 1 is a view of the lumbar orthosis showing the pocket and the rigid backplate.

FIG. 2 is a semi-transparent view of the lumbar orthosis laid flat.

FIG. 3 is a view of the lumbar orthosis being worn by a patient, showing the magnets.

FIG. 4 is a view of an alternate embodiment of the lumbar orthosis.

### DETAILED DESCRIPTION OF THE PREFERRED EMBODIMENT

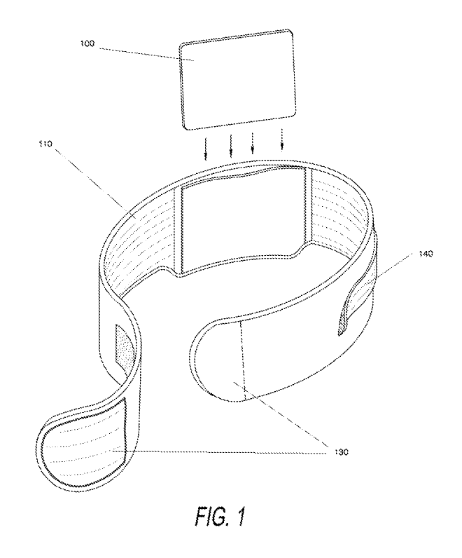

FIG. 1 shows a view of the preferred embodiment of the lumbar orthosis. Rigid backplate 100 is inserted into a pocket on belt 110. The rigid backplate 100 is preferably made of a thermoplastic material typically used for orthoses, as is well known in the art of orthotics. The material can be heated and molded to the patient's individual contours for greater comfort and better support.

Belt 110 is fastened around the patient's waist by means of primary fastener 130. This primary fastener 130 is preferably a Velcro fastener to allow easy attachment and removal without too much bulk. Other types of fasteners may also be used. In the preferred embodiment of the invention, the belt is anywhere between 6″ and 8″ wide, and may be made of a heat-retention fabric to provide therapeutic heat to the injured area.

Supplementary fasteners 140 are located at the patient's sides. These fasteners allow the patient to tighten or loosen the orthosis without disturbing the primary fastener. Such fasteners may, as shown in the Figure, be straps that are attached to the belt on one end and that have a Velcro fastener on the other end that can be removably attached to the belt at several possible points along its length. Other fastening means, such as snaps or hooks, may also be used to attach the other end of the supplementary fastener 140 to the belt 110. In an alternate embodiment, the supplementary fasteners 140 are laces that are laced through holes in the belt and allow the patient to tighten the belt by pulling on the laces.

Primary fastener 130 includes a pocket into which a semi-rigid front plate can be inserted. This provides extra support and compression for the patient's torso, as well as greater stability.

The sides of the belt 110 may be made out of breathable elastic to provide greater comfort for the patient and to prevent sweating.

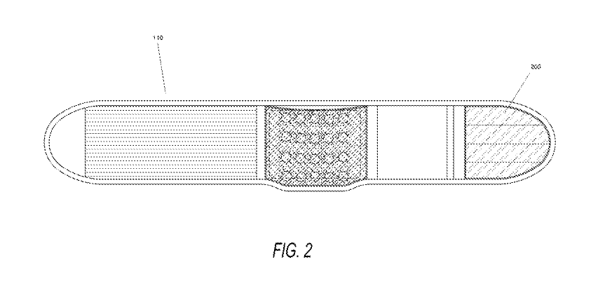

FIG. 2 shows a semi-transparent view of the lumbar orthosis lying flat. A semi-rigid front plate 200 is inserted into a pocket on belt 110. The semi-rigid front plate provides extra stability and compression; it is preferably made of a thin sheet of flexible plastic that is flexible enough to be comfortable for the patient while being rigid enough to provide some additional support for the patient's torso from the front. This is intended for patients with more severe back problems where just the rigid backplate is insufficient.

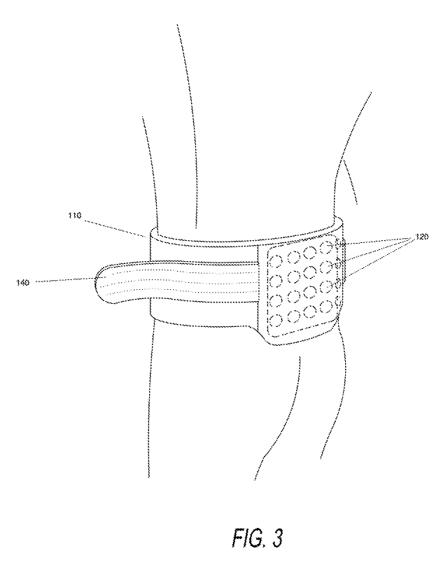

FIG. 3 shows the lumbar orthosis being worn by a patient. Permanent magnets 120 are attached to the belt 110 in the patient's lumbar area. The magnets provide a permanent magnetic field that improves circulation and assists in healing. The magnets may be placed in any pattern, and any quantity or shape of magnets may be used, as long as the magnetic field they provide is sufficient for a therapeutic effect. In the preferred embodiment, an array of disc-shaped magnets are attached to the belt in the patient's lumbar area, and placed on top of the rigid backplate so as to not interfere with the fit of the backplate. In alternate embodiments, the magnets may also be attached to the rigid backplate or embedded within the rigid backplate, or embedded within the pocket material. In other embodiments, the backplate itself may be magnetic.

In another embodiment of the invention, permanent magnets are located in both the lumbar area and the abdominal area. The preferred number of magnets for that embodiment is 20 permanent magnets in the lumbar area and 6 permanent magnets in the abdominal area. This creates a three-dimensional magnetic field that has a greater therapeutic value for the patient.

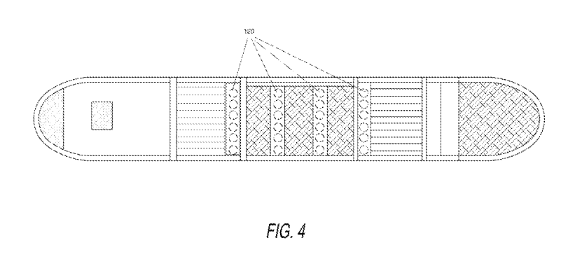

FIG. 4 shows a flat view of an alternate embodiment of the lumbar orthosis. In this embodiment, permanent magnets 120 are arranged in vertical lines rather than a single rectangular array. This provides greater comfort to the patient by making the orthosis more flexible without sacrificing the beneficial advantages of the magnetic field.

1. A lumbar orthosis, comprising:
a belt;
a rigid backplate molded to the patient's lumbar area and attached to the belt; and
at least one permanent magnet located on the patient's lumbar area.

2. The lumbar orthosis of claim 1, further comprising a front plate located on the patient's abdominal area.

3. The lumbar orthosis of claim 2, where the front plate is semi-rigid.

4. The lumbar orthosis of claim 1, further comprising at least one permanent magnet located on the patient's abdominal area.

5. The lumbar orthosis of claim 1, where at least one permanent magnet is attached to the rigid backplate.

6. The lumbar orthosis of claim 1, where at least one permanent magnet is attached to the belt.

7. The lumbar orthosis of claim 1, where at least one permanent magnet is located on the outside of the rigid backplate.

8. The lumbar orthosis of claim 1, where at least one permanent magnet is located on the inside of the rigid backplate.

9. The lumbar orthosis of claim 1, where at least one permanent magnet is embedded within the material of the rigid backplate.

10. The lumbar orthosis of claim 1, where the belt is made of breathable elastic.

11. The lumbar orthosis of claim 1, where the rigid backplate is removably inserted into a pocket on the belt.

12. The lumbar orthosis of claim 2, where the front plate is removably inserted into a pocket on the belt.

13. The lumbar orthosis of claim 1, where the permanent magnets are arranged in a rectangular array on top of the rigid backplate.

14. The lumbar orthosis of claim 1, where the permanent magnets are arranged in vertical linear arrays on top of the rigid backplate.

15. The lumbar orthosis of claim 4, further comprising a front plate located on the patient's abdominal area.

16. The lumbar orthosis of claim 15, where the permanent magnets are arranged in a rectangular array on top of the front plate.

17. The lumbar orthosis of claim 15, where the permanent magnets are arranged in vertical linear arrays on top of the front plate.

18. The lumbar orthosis of claim 15, where at least one permanent magnet is embedded within the material of the front plate.

19. The lumbar orthosis of claim 15, where at least one permanent magnet is located on the outside of the front plate.

20. The lumbar orthosis of claim 15, where at least one permanent magnet is located on the inside of the front plate.

21. The lumbar orthosis of claim 1, where the belt is made of a heat-retention fabric.

22. The lumbar orthosis of claim 1, further comprising:
fastening adjustment means on the sides of the orthosis such that the tightness of the orthosis can be adjusted without removing the orthosis.

23. A lumbar orthosis, comprising:
a belt;
a rigid backplate molded to the patient's lumbar area and attached to the belt,
where the rigid backplate emits a magnetic field.

24. The lumbar orthosis of claim 23, further comprising a semi-rigid front plate attached to the belt in the patient's abdominal area.

25. The lumbar orthosis of claim 24, where the semi-rigid front plate emits a magnetic field.
